(19) United States
(12) Patent Application Publication
(10) Pub. No.: US 2006/0172402 A1
(43) Pub. Date: Aug. 3, 2006

(54) Production of vanillin in microbial cells

(76) Inventors: Daphna J Havkin-Krenkel, North Brunswick, NJ (US); Gerben Zylstra, Roosevelt, NJ (US); Chaim Frenkel, North Brunswick, NJ (US); Faith Belanger, East Brunswick, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/532,464

(22) PCT Filed: Oct. 23, 2003
(86) PCT No.: PCT/US03/34011

§ 371 Date: Nov. 7, 2005

Related U.S. Application Data
(60) Provisional application No. 60/412,649, filed on Oct. 23, 2002.

(51) Int. Cl. C12P 7/24; C12N 9/10; C12N 1/21; C12N 1/18

(52) U.S. Cl. 435/147; 435/252.33; 435/252.34; 435/254.21; 435/254.23; 435/193

(57) ABSTRACT

Transgenic microorganisms that produce vanillin when provided with caffeic acid or an esterified or other derivative thereof are disclosed. The organisms are transformed with expressible nucleic acid sequences encoding (1) a 3-0-methyltransferase, preferably from a plant source, which converts caffeic acid to ferulic acid and (2) either a eukaryotic (preferably plant) non-oxidative chain-shortening enzyme or a bacterial CoA ligase and enoyl-CoA hydratase/lyase enzymatic system, either of which converts ferulic acid to vanillin. Methods of making vanillin in the transgenic microorganisms are also disclosed.

Benefit is claimed of U.S. Provisional Application No. 60/412,649, filed Oct. 23, 2002, the entirety of which is incorporated by reference herein.

### FIELD OF THE INVENTION

This invention relates to the field of microbial genetic engineering to produce high-value food and nutraceutical substances. In particular, this invention provides novel transgenic microbial cells that produce vanillin.

### BACKGROUND OF THE INVENTION

Various patents and publications are referred to throughout the specification. Each of these is incorporated by reference herein in its entirety.

Vanillin is the major principle flavor ingredient in vanilla extract and is also noted as a nutraceutical because of its anti-oxidant and antimicrobial properties. Vanillin can be used as a masking agent for undesirable flavors of other nutraceuticals. Vanilla extract is obtained from cured vanilla beans, the bean-like pod produced by Vanilla planifolia, a tropical climbing orchid.

Vanilla extract is widely used as a flavor by the food and beverage industry, and is used increasingly in perfumes. Because of the ever-increasing demand for natural food ingredients, natural vanilla extract produced from vanilla beans is presently the most desirable form of vanilla. The areas of the world capable of supporting vanilla cultivation are limited, due to its requirement for a warm, moist and tropical climate with frequent, but not excessive, rain and moderate sunlight. The primary growing region for vanilla is around the Indian Ocean, in Madagascar, Comoros, Reunion and Indonesia.

The production of vanilla beans is a lengthy process that is highly dependent on suitable soil and weather conditions. Beans (pod-like fruit) are produced after 4-5 years of cultivation. Flowers must be hand-pollinated, and fruit production takes about 8-10 months. The characteristic flavor and aroma develops in the fruit after a process called “curing,” lasting an additional 3-6 months. For a complete review of the vanilla growing and curing process, see D. Havkin Frenkel & R. Dorn, “Vanilla,” Chapter 4 in Spices: Flavor Chemistry and Antioxidant Properties, (Eds. Risch & Ho), American Chemical Society, Washington, 1997.

Vanillin is also produced chemically by molecular breakage of curcumin, eugenol or piperin. However, vanillin produced by this method can be labeled as a natural flavor only in non-vanilla flavors. Vanillin chemically synthesized from guaiacol is consumed at a rate of about 2,500 tons per year in the United States for the food and beverage industry. Though less expensive than natural vanillin, vanillin produced by chemical synthesis or breakage can be undesirable due to the market's current preference for natural food ingredients.

Interest has focused recently on plant cell and tissue culture as an approach to control quality and yield of vanilla production and to solve some of the agronomic problems associated with growing vanilla. Another possible means for producing vanillin is through the use of microorganisms engineered to possess the requisite complement of vanillin biosynthetic enzymes.

Several C₆-C₃ source compounds, mostly eugenol and ferulic acid, are currently in use in conjunction with fermentation technologies, for the biotechnological production of vanillin (Benz, 1996, Biotechnological production of vanillin. In: A J Taylor and D S Mottram, Eds, Flavour science—Recent Development, The Royal Society of Chemistry, Cambridge, UK, pp 111-117). Eugenol, a major aromatic constituent in clove oil, is converted by a Pseudomonas strain to ferulic acid through successive steps entailing formation of coniferyl alcohol, coniferyl aldehyde and, finally, ferulic acid. Ferulic acid is present also in cereal crops where the compound is esterified to arabinose moieties comprising around 0.4 to 3.0% of the cell wall material (Walton et al., 2000, Curr. Op. Biotechnol. 11: 490-496). Ferulic acid may be released from the cell wall matrix with the use of strong alkali or by enzymatic cleavage of the wall material using cinnamoyl esterase in combination with cell wall hydrolyzing enzymes (Williamson et al., 1998, Microbiology 144: 2011-2023). Such processes are expensive and time consuming, and can require specialized equipment.

Moreover, bioconversion of ferulic acid to produce vanillin, as opposed to undesired by-products such as vanillic acid, heretofore has not been a straightforward process. Although ferulate is readily metabolized by various microbial systems, the end product is mostly vanillic acid (Dignum & Verpoorte, 2001, Food Rev. Int. 17: 199-219).

It appears that the mode of degradation of a three-carbon side chain of a hydroxycinnamic acid derivative, eugenol or ferulic acid for instance, to a single carbon moiety determines the metabolic fate of phenylpropanoid compounds. There are several reports on the in vitro chain shortening-catalyzed degradation of C₆-C₃ to C₆-C₁ compounds, such as benzoic acids and aldehydes, from hydroxycinnamic acids. One study on the synthesis of 4-hydroxybenzoate in L. erythrorhizon indicates that the pathway entails oxidation and cleavage of 4-coumaroyl CoA to 4-hydroxybenzoyl CoA and acetyl CoA in a thiolase type reaction with requirement for NAD (Lüscher and Heide, 1994, Plant Physiol 106: 271-279). This mode of enzyme action, involving oxidative chain shortening, may account for the formation of vanillic acid as an oxidative cleavage product from ferulic acid, instead of the sought-after vanillin.

Microorganisms capable of utilizing abundant and inexpensive starting materials to produce vanillin in a straightforward manner, without unwanted by-products are currently not available. Thus, a need exists for their creation and development.

### SUMMARY OF THE INVENTION

The present invention features a transgenic microorganism that produces vanillin when provided with caffeic acid or derivative thereof of esterified coumaric acid. The organism is transformed with expressible nucleic acid sequences encoding (1) a 3-O-methyltransferase, preferably from a plant source, which converts caffeic acid to ferulic acid and (2) either a eukaryotic (preferably plant) non-oxidative chain-shortening enzyme or a bacterial CoA ligase and enoyl-CoA hydratase/lyase enzymatic system, either of which converts ferulic acid to vanillin. In one embodiment, the microorganism comprises, naturally or via recombinant means, an expressible nucleic acid molecule encoding an esterase that converts caffeic acid esters (e.g., cichoric acid, rosmarinic acid or chlorogenic acid) to caffeic acid.

In one embodiment, the transgenic microorganism is a procaryote, such as E. coli, Pseudomonas or any other prokaryotic microorganism that can be transformed and used for expression of foreign proteins. In another embodiment, the transgenic microorganism is a eucaryote, such as the yeasts Saccharomyces cerevisiae or, in a preferred embodiment, Pichia pastoris. The microorganism preferably one that does not degrade or further metabolize vanillin, once it is produced.

The present invention also features a method for producing vanillin, which comprises: (a) providing a transgenic organism that produces vanillin when provided with caffeic acid or an esterified derivative thereof, as described above; (b) culturing the transgenic organism in the presence of the caffeic acid or derivative thereof, under conditions whereby the transgenic organism produces vanillin; and (c) recovering the vanillin from the culture.

Another aspect of the invention features an O-methyltransferase from Vanilla planifolia that catalyzes methylation of substrates selected from the group consisting of 5-OH-ferulic acid ethyl ester, caffeic acid ethyl ester, caffeoyl aldehyde, 5-OH-coniferaldehyde, 5-OH-ferulic acid, 3,4-dihydroxybenzaldehyde and caffeic acid. In one embodiment, the enzyme has an amino acid sequence at least 90% identical to SEQ ID NO:2, and more specifically comprises amino acid SEQ ID NO:2.

Also featured is an isolated nucleic acid molecule that encodes the O-methyltransferase described above. In one embodiment, the nucleic acid encodes a polypeptide having an amino acid sequence at least 90% identical to SEQ ID NO:2 and more specifically encodes a polypeptide having SEQ ID NO:2. In an exemplary embodiment, the nucleic acid molecule has a sequence comprising SEQ ID NO:1.

Additional features and advantages of the present invention will be understood by reference to the drawings, detailed description and examples that follow.

### BRIEF DESCRIPTION OF THE DRAWINGS

FIG. 1. Schematic diagram showing the biotransformation of cichoric acid to vanillin.

FIG. 2. Schematic diagram showing the biotransformation of rosmarinic acid to vanillin.

### DETAILED DESCRIPTION OF ILLUSTRATIVE EMBODIMENTS

I. Definitions

Various terms relating to the biological molecules of the present invention are used hereinabove and also throughout the specification and claims.

With reference to nucleic acid molecules the term “isolated nucleic acid” or “isolated polynucleotide” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule. With respect to RNA molecule, the term “isolated nucleic acid” primly refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule. Alternatively, this term may refer to a protein which has been sufficiently separated from other proteins with which it would naturally be associated, so as to exist in “substantially pure” form.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

The term “enzyme” refers to a protein having enzymatic activity. As used herein, the term enzyme may refer to the singular or plural, in instances where two or more enzymes form an enzymatic system to convert one substance into another.

“Antibodies” as used herein includes polyclonal and monoclonal antibodies, chimeric, single chain, and humanized antibodies, as well as Fab fragments, including the products of an Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence; as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring, such as an allelic variant, or it may be a variant that is not known to occur naturally. Non-naturally occurring variants of polynucleotides and polypeptides may be made by mutagenesis techniques or by direct synthesis.

The term “substantially the same” refers to nucleic acid or amino acid sequences having sequence variations that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “identity” or “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

“Identity” and “similarity” can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the DNAstar system (Madison, Wis.) is used to align sequence fragments of genomic DNA sequences. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by that program may also be used to compare sequence identity and similarity.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforiiing DNA” or “transgene”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The following sections set forth the general procedures involved in practicing the present invention. To the extent that specific materials are mentioned, it is merely for the purpose of illustration, and is not intended to limit the invention. Unless otherwise specified, general biochemical and molecular biological procedures, such as those set forth in Sambrook et al., Molecular Cloning, Cold Spring Harbor Laboratory (1989) or Ausubel et al. (eds), Current Protocols in Molecular Biology, John Wiley & Sons (2003) are used.

II. Description

In an effort to develop alternative methods for producing high quality vanillin in an economically feasible way, the inventors have devised a biotransformative pathway that can be engineered into selected species of microorganisms, which results in production of vanillin from a readily available and inexpensive starting material, which is caffeic acid or a substance that readily produces caffeic acid, such as cichoric acid, rosmarinic acid or chlorogenic acid (sometimes referred to collectively herein as “caffeic acid derivatives”).

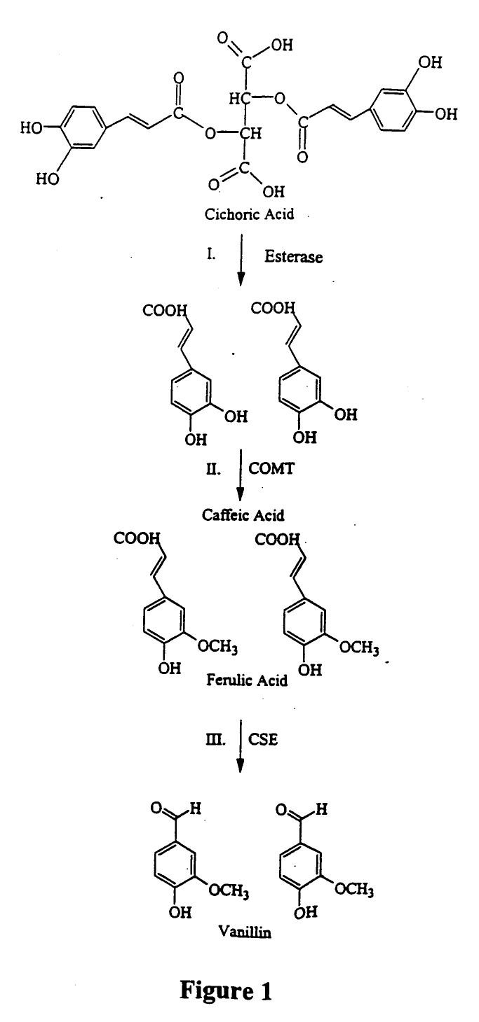

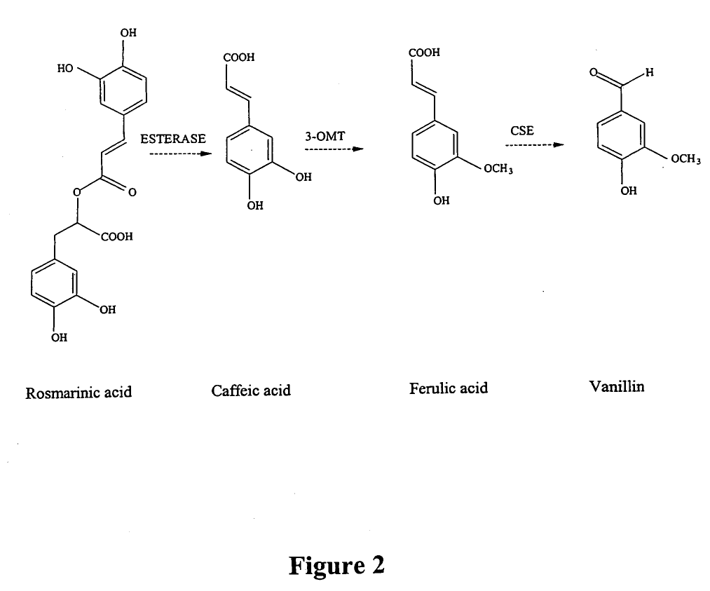

Representative schemes for vanillin biosynthesis in accordance with the invention are outlined in FIGS. 1 and 2. The schemes indicate that caffeic acid, obtained in the native form or by hydrolysis of caffeic acid esters, is methylated to ferulic acid by 3-O-methyltransferase, a readily available gene product, which has also been cloned from V. planifolia itself. In a preferred embodiment of the present invention, ferulic acid is thereafter converted in one-step process to vanillin by the action of a non-oxidative chain-shortening enzyme, exemplified by the V. planifolia 4-hydroxybenzaldahyde synthase (4-HBS) disclosed in U.S. Published Application No. 2003/0070188 A1 (April 2003) to Havkin-Frenkel et al. An alternative embodiment uses bacterial enzymes in a two-step non-oxidative process to convert ferulic acid to vanillin.

Advantageous to the present invention, caffeic acid or caffeic acid derivatives are abundant in several plant species. These compounds are readily hydrolyzed by esterase action, resulting in the release of caffeic acid (Nusslein et al. 2000, J. Nat. Prod. 63: 1615-161 S). Hence, hydrolytically produced caffeic acid, combined with the content of native caffeic acid, can yield 10 to 15% free caffeic acid on dry weight basis. Because caffeic acid or caffeic acid derivatives are present in plant tissues in a free form and because these compounds are readily extracted (e.g., by ethanol), these materials offer an important advantage as source compounds. By comparison, the use of ferulic acid itself as source material is predicated on enzymatic release of the compound from its bound form to cell walls. This process is complex, leading to impurities and, importantly, is costly. The method of the present invention averts these problems by making use of abundant and readily extractable caffeic acid or caffeic acid derivatives in plant tissues and by a direct methylation of caffeic acid to form ferulic acid. The subsequent conversion of ferulate to vanillin by a non-oxidative chain shortening enzyme also avoids the inadvertent conversion of C₆-C₃ compound to vanillic acid or other unintended end products; a problem encountered in other production systems.

Natural sources for caffeic acid and derivatives thereof include, but are not limited to, Echinacea spp. and other species in the mint family, and may further include any plant species that contain the compounds. In a preferred embodiment, cichoric acid is obtained from Echinacea spp. Table 1 shows other plant sources of caffeic acid or its derivatives. In addition to those listed in Table 1, plant species that contain caffeic acid or derivatives suitable for use in the present invention include, but are not limited to, liquorice (Glycyrrhiza glabra, G. inflata, G. uralensis), oregano (Origanum compactum and other species), sage (Salvia fruticosa), carrot (Daucus carota), fennel (Foeniculum vulgare) and artichoke (Cynara cardunculus).

TABLE 1
Abundance of Caffeic acid and Caffeic
acid derivative in some plant species

| | Caffeic | Rosmarinic ¹ | Chicoric ² |
|---|---|---|---|
| Plant Species | Acid | Acid | Acid |
| | % Dry Weight ³ | | |
| Ocimuin basilicum | 0.5-2.5 | 5.0-8.0 | |
| Agastache sp. | 1.0-2.0 | 8.0-10.0 | |
| Echinacea purpurea | 3.6 | | 16.7 |
| Mentha sp. | 1.0-2.0 | 2.0-15.0 | |
| Rosmarinus officianalis | 0.1-2.0 | 4.0-10.0 | |

¹ Cinnamic acid, 3,4-dihydroxy-, 2-ester with 3-(3,4-dihydroxyphenyl) lactic acid
² Tartaric acid, bis (3,4-dihydroxycinnamate)
³ Value were obtained by HPLC analysis.

The biotransformation of cichoric acid to vanillin is shown schematically in FIG. 1. A similar biotransformation is accomplished using rosmarinic acid, as shown in FIG. 2. Cichoric acid, rosmarinic acid and chlorogenic acid (5-caffeolylquinic acid) are all esters of caffeic acid, and can be converted to caffeic acid in a similar manner. Other caffeic acid esters that can be utilized as caffeic acid sources include, but are not limited to, 1-caffeolylquinic acid and 1,3-dicaffeolylquinic acid (cynarin). Hydrolysis of caffeic acid esters can be accomplished with heat, pressure and mild alkaline solution, or enzymatically by esterases, which is a preferred embodiment. Esterases suitable for catalyzing the conversion of caffeic acid esters to caffeic acid are known in the art and are present in plants, animals and many microorganisms. In the latter instance, therefore, the esterases often need not be engineered into such microorganisms because they exist there naturally. In a preferred embodiment, a microorganism naturally capable of producing caffeic acid from esters thereof is utilized in the present invention.

Caffeic acid is methylated to produce ferulic acid, using a 3-O-methyltransferase obtainable from numerous plant sources, among other organisms. This enzyme catalyzes a methylation at position 3 on the ring (and may also methylate position 5 if it is hydroxylated). Examples of suitable 3-methyltransferases include, but are not limited to (GenBank Accession Numbers follow each listed source organism): Catharanthus roseus, AY028439; Clarkia breweri, AF006009; Coffea canephora, AF454631; Eucalyptus gunnii, X74814; Festuca arundinacea, AF153825; Hordeum vulgare, U54767; Hordeum vulgare, AB086416; Lolium perenne, AF010291; Medicago sativa, M63853; Nicotiana tabacum class I, X74452; Nicotiana tabacum class II X74452; Ocimum basilicum, AF154918; Populus tremuloides, X62096; Prunus amygdalus, X83217; Saccharum officinarum, AJ231133; Sorghum bicolor, AY217766; Thalictrum tuberosum, AF064696; Triticum aestivum, AY226581; and Zea mays, M73235. Nucleic acid and deduced amino acid sequences set forth in the aforementioned Accessions are each incorporated by reference herein in their entireties. Preferred for use is the 3-O-methyltrasferase from Medicago sativa (alfalfa). Also preferred for use is the 3-O-methyltransferase from Vanilla planifolia. A cDNA sequence (SEQ ID NO:1) and deduced amino acid sequence (SEQ ID NO:2) of this enzyme are shown in the Sequence Listing that forms part of this document.

In one embodiment, ferulic acid is converted to vanillin using a eukaryotic non-oxidative-chain shortening enzyme, such as the plant-derived 4-HBS described in U.S. Published Application No. 2003/0070188 A1 to Havkin-Frenkel et al. (2003). Any similar eukaryotic aldehyde synthase that acts as a non-oxidative chain-shortening enzyme may also be utilized. Conversion of ferulic acid to vanillin by non-oxidative means offers the advantage of reducing or eliminating formation of undesired vanillic acid, as discussed above.

In an alternative embodiment, ferulic acid is converted to vanillin using a bacterial chain shortening enzyme system, enoyl-SCoA hydratase/lyase (Gasson et al., 1998, J. Biol. Chem. 273: 4163-4170). The bacterial process is a two-step enzymatic process, involving first a CoA ligase (an enzyme found in eukaryotes, see, e.g., Gross et al., 1973, FEBS Lett. 31: 283-287, as well as bacteria) to activate ferulate to the CoA derivative and then a hydratase/lyase to enzymatically cleave the double bond, releasing vanillin and acetyl-CoA. This enzyme system has been demonstrated to catalyze the conversion of ferulic acid to vanillin. It is found in a number of bacteria, including but not limited to Pseudomonas florescens (Civolani et al., 2000, Appl Environ Microbiol. 66: 2311-2317; Narbad and Gasson, 1998, Microbiology 144: 1397-1405), Pseudomonas putida (Venturi et al., 1998, Microbiology 144: 965-973), other Pseudomonas species (Overhage et al., 1999, Appl Microbiol Biotechnol. 52: S20-828; Overhage and Steinbuchel, 1999, Appl Environ Microbiol. 65: 4837-47) and Nocardia spp. (Li and Rosazza, 2000, Appl. Environ. Microbiol. 66: 684-687).

In certain instances, enoyl-SCoA hydratase/lyase may utilize caffeic acid as a substrate to form 3,4-dihydroxy benzaldehyde. This product also may be converted to vanillin through the action of the above-described 3-O-methyltransferases.

Expression vectors comprising DNA that encodes the aforementioned enzymes are introduced into a selected microorganism. Preferably, a microorganism that is amenable to genetic manipulation is utilized. In addition, it is preferred that the microorganism is not capable of degrading or further metabolizing the end product, vanillin. Suitable microorganisms for practice of the invention include, but are not limited to, E. coli and Pseudomonas spp. as model procaryotic expression systems and yeast such as Saccharomyces cerevisiae or Pichia pastoris as model eucaryotic expression systems. Vectors and systems for transforming these and other organisms are well known in the art.

After the microorganism has been engineered to express all enzymes necessary for the conversion of the caffeic acid or its derivatives to vanillin, production of vanillin is accomplished as follows: (1) grow the engineered microorganism in a suitable culture medium; (2) add the selected caffeic acid or derivative to the culture medium; (3) grow the culture for a time, and under conditions to enable production of vanillin, which preferably, but not necessarily, is secreted into the medium; and (4) recover the vanillin from the cells or medium. Vanillin can be purified from a solution by well-established methods (e.g., Priefert et al., 2001, Appl. Microbiol. Biotechnol. 56: 296-314; Klinke et al., 2002, Bioresource Technology 82: 15-26), used in vanillin manufacturing from lignin, for instance. Examples include vanillin volatilization from solutions above 80° C. and crystallization from saturated solutions.

The present invention also provides a novel multifunctional methyltransferase from Vanilla planifolia, and its encoding nucleic acid, both of which are useful in the practice of the present invention and for other purposes. This enzyme, referred to herein as “vpOMT” is capable of catalyzing the conversion of caffeic acid to ferulic acid, and also the conversion of 3,4-dihydroxybenzaldehyde to vanillin. Details of the isolation and characterization of vpOMT, and the cloning of a cDNA encoding vpOMT, are set forth in the examples.

A cDNA encoding vpOMT is set forth herein as SEQ ID NO:1, and its encoded protein is set forth herein as SEQ ID NO:2. Although this particular cDNA and polypeptide are described and exemplified herein, this invention is intended to encompass proteins from other Vanilla cultivars and species that are sufficiently similar to be used interchangeably with the characterized vpOMT for the purposes described herein.

Accordingly, considered in terms of their sequences, vpOMT-encoding nucleic acids of the invention include allelic variants and natural mutants of SEQ ID NO:1, which are likely to be found in different varieties of V. planifolia and Vanilla, and homologs of SEQ ID NO:1 likely to be found in different plant species. Because such variants and homologs are expected to possess certain differences in nucleotide and amino acid sequence, this invention provides an isolated vpOMT-encoding nucleic acid molecule that encodes a vpOMT polypeptide having at least about 90% (and, with increasing order of preference, 91%, 92%, 93%, 94%, 95%, 96%, 97%, 98% and 99%) identity with SEQ ID NO:2, with a corresponding level of nucleotide sequence identity with respect to SEQ ID NO:1. Because of the natural sequence variation likely to exist among vpOMT enzymes and the genes encoding them in different plant varieties and species, one skilled in the art would expect to find this level of variation, while still maintaining the unique properties of the vpOMT of the present invention. Such an expectation is due in part to the degeneracy of the genetic code, as well as to the known evolutionary success of conservative amino acid sequence variations, which do not appreciably alter the nature of the encoded protein. Accordingly, such variants and homologs are considered substantially the same as one another and are included within the scope of the present invention.

VpOMT-encoding nucleic acid molecules of the invention may be prepared by two general methods: (1) they may be synthesized from appropriate nucleotide triphosphates, or (2) they may be isolated from biological sources. Both methods utilize protocols well known in the art.

The availability of nucleotide sequence information, such as the cDNA having SEQ ID NO:1, enables preparation of an isolated nucleic acid molecule of the invention by oligonucleotide synthesis. Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC).

VpOMT genes also may be isolated from appropriate biological sources using methods known in the art. Nucleic acids having the appropriate level sequence homology with part or all of SEQ ID NO:1 may be identified by using hybridization and washing conditions of appropriate stringency. For example, hybridizations may be performed, according to the method of Sambrook et al., using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 2×SSC and 0.1% SDS; (4) 2 hours at 45-55° C. in 2×SSC and 0.1% SDS, changing the solution every 30 minutes.

One common formula for calculating the stringency conditions required to achieve hybridization between nucleic acid molecules of a specified sequence homology (Sambrook et al., 1989):

Tₘ=81.5EC+16.6 Log[Na+]+0.41(% G+C)−0.63 (% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and wash in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and wash in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and wash in 0.1×SSC and 0.5% SDS at 65 C for 15 minutes.

Nucleic acids of the present invention may be maintained as DNA in any convenient cloning vector. In a preferred embodiment, clones are maintained in plasmid cloning/expression vector, such as pGEM-T (Promega Biotech, Madison, Wis. or pBluescript (Stratagene, La Jolla, Calif.), either of which is propagated in a suitable E. coli host cell.

VpOMT nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of SEQ ID NO:1.

VpOMT polypeptides may be prepared in a variety of ways, according to known methods. In one embodiment the protein is purified from appropriate sources, e.g., plant tissue as described in the examples.

Alternatively, the availability of nucleic acid molecules encoding the polypeptides enables production of the proteins using ill vitro expression methods known in the art. For example, vpOMT may be produced by expression in a suitable procaryotic or eucaryotic system. Part or all of a DNA molecule, such as the cDNA having SEQ ID NO:1, may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

Polyclonal or monoclonal antibodies directed toward any of the peptides encoded by vpOMT may be prepared according to standard methods. Monoclonal antibodies may be prepared according to general methods of Köhler and Milstein, following standard protocols.

The following examples are provided to describe the invention in greater detail. They are intended to illustrate, not to limit, the invention.

### EXAMPLE 1

### Methods for Isolation and Purification of vpOMT Protein and cDNA

Plant Material

Tissue cultures of V. planifolia were initiated and maintained as described by Podstolski et al. (2002, Phytochemistry 61: 611-620). Plants of V. planifolia were maintained in the greenhouse and were the source of stem, leaf, and root tissues. Green V. planifolia pods at different stages of development were obtained from Indonesia.

Enzyme Extraction and Assay

Preparation of crude protein extracts of the V. planifolia pods and tissue cultures grown in liquid media was modified from that described by Wang et al. (1997, Plant Physiol. 114: 213-221). For determining the presence of DOMT activity, 3 g tissue was homogenized in 6 ml of 50 mM BisTris-HCl, pH 6.9, 10 mM 2-mercaptoethanol, 5 mM Na₂S₂O₅, 1% (w/v) PVP-40, 1 mM phenylmethanesulfonyl fluoride (PMSF), and 10% (v/v) glycerol. The homogenate was filtered through cheesecloth and centrifuged 15 min at 10,000 g at 4° C.

Protein concentrations were determined using the Bio-Rad protein assay reagent (Bio-Rad, Richmond, Calif.) with bovine serum albumin as a standard.

O-Methyltransferase assays were as described by Wang et al. (1997, supra). Assays were done in 50 μl volumes and were composed of 10 μl assay buffer (250 mM Tris-HCl, pH 7.5, 10 mM DTT), 1 μl of 50 mM substrate, 10 μl enzyme (crude extracts or fractions from partial purification), and 1 μl of S-[methyl-¹⁴C]adenosyl-L-methionine (SAM) (59 mCi/mmol)(Amersham Pharmacia Biotech, Buckinghamshire, England), and 28 μl water. The samples were incubated at 30° C. for 30 min, after which the reactions were stopped by adding 2.5 μl of 6 M HCl. [¹⁴C]SAM was separated from the radiolabeled methylated product by extraction with 100 μl ethyl acetate. Twenty μl of the organic phase containing the labeled product was used for liquid scintillation counting. The counts per min were converted to pkat (picomoles of product produced per second), based on the specific activity of the substrate and the efficiency of the scintillation counter.

For determination of the kinetic parameters, the reaction conditions were modified to include 2 μl [¹⁴C]SAM, 100 μM unlabelled SAM, and 3 μg of the purified recombinant protein expressed in E. coli. Substrate concentrations ranged from 0.001 mM to 4 mM. All reactions were done in duplicate. Vmax and Km were calculated from nonlinear regressions of the Michaelis-Menton plots using the program Prism 4 (GraphPad Software, Inc., San Diego, Calif.).

Thin Layer Chromatography

The identity of vanillin as the labeled reaction product following methylation of 3,4-dihydroxybenzaldehyde was confirmed by TLC analysis. Twenty μl aliquots of the organic extract were spotted onto a 20 cm×20 cm silica gel 60 precoated TLC plate (EM Industries, Inc, Gibbstown, N.J.). Twenty μl each of 10 mM vanillin, 10 mM 3,4-dihydroxybenzaldehyde and a mixture of both were also spotted as standards. The plate was developed in a solvent system of chloroform:acetic acid (9:1, v/v). To visualize the standards following chromatography, the plate was allowed to dry and examined under UV light. The region of the plate from the reaction product that corresponded to the position of standard vanillin was scraped into scintillation vials and counted.

Partial Purification of V. planifolia OMT

For protein purification, a crude extract of the tissue culture was prepared by homogenizing in 10 volumes fresh weight of the extraction buffer. Partial purification of DOMT activity from the crude extract on an adenosine-agarose affinity column was modified from that described by Wang and Pichersky (1998, Arch. Biochem, Biophys. 349:153-160). A 1 ml adenosine-agarose (Sigma, St. Louis, Mo.) column was prepared as previously described (Attieh et al., 1995, J. Biol. Chem. 270: 9250-9257). Ten ml of tissue culture crude extract was applied to the adenosine-agarose column. The column was washed with 6 ml 50 mM Bis-Tris, pH 6.9, 10 mM 2-mercaptoethanol, 10% glycerol followed by elution with 10 ml wash buffer containing 2.5 mM adenosine. One ml fractions were collected and assayed for DOMT and COMT activities. Fractions containing activity were combined and concentrated using Microcon YM30 devices (Amicon, Beverly, Mass.).

PCR Amplifications of O-methyltransferase cDNA Fragment

Degenerate oligonucleotide primers for PCR were designed based on conserved sequences in COMTs from other plant species. The amino acid sequences encoded by the primers were VLMESWY and HVGGDMF, respectively.

The degenerate oligonucleotide primers were used in PCR amplification of the cDNA library prepared from the V. planifolia tissue cultures. PCR reactions were carried out using the Elongase Amplification System (Invitrogen, Carlsbad, Calif.). The 100 μl reactions contained 60 mM Tris-SO₄, pH 9.1, 18 mM (NH₄)₂SO₄, 1.5 mM MgSO₄, 200 μM each dNTP, 3 μg of each oligonucleotide, and 2 μl Elongase enzyme mix. PCR was carried out in a GeneAmp 9600 thermocycler (Perkin Elmer Life Sciences, Boston, Mass.). Touchdown PCR cycling parameters were used. Initial denaturation was conducted at 94° C. for 30 s. Cycle 1 consisted of denaturation at 94° C. for 30 s, annealing at 66° C. for 30 s, and extension at 68° C. for 2 min. Every two subsequent cycles, the annealing temperature was decreased by 1° C. until 56° C. was reached. An additional 30 cycles at an annealing temperature of 56° C. were performed, followed by a final extension at 68° C. for 10 min. PCR products were resolved on a 1.2% (w/v) agarose gel, and a single band of about 350 bp was detected. The DNA band was excised and purified using a commercial kit (QIAquick Gel Extraction Kit, Qiagen USA). The purified band was ligated into the pGEM-T Easy vector (Promega, Madison, Wis.) and transformed into JM109 E. coli competent cells. Plasmids were purified from E. coli transformants using a commercial kit (QIAprep Spin Miniprep Kit, Qiagen) and sequenced using SP6 and T7 primers.

cDNA Library Screening

A cDNA library was constructed by Stratagene (LaJolla, Calif.) in the λ ZAP-Express vector using poly(A⁺) RNA from V. planifolia tissue culture. Four hundred and fifty thousand plaque-forming units were screened using the 350 bp PCR clone as probe. The cloned 350 bp fragment was labeled with [α³²P]dCTP using a commercial kit (Prime-It II Random Primer Labeling Kit, Stratagene).

The plaque lifts were prehybridized at 42° C. in 50% (v/v) formamide, 5×SSC, 5× Denhardt's solution [1× Denhardt's solution is 0.02% (w/v) Ficoll, 0.02% (w/v) PVP, 0.02% (w/v) BSA], 50 mM sodium phosphate, pH 6.8, 1% (w/v) SDS, 100 μg ml⁻¹ calf thymus DNA, and 2.5% (w/v) dextran sulfate. The hybridization solution was 5×10⁵ cpm ml⁻¹ of ³²P-labeled fragment, 50% (v/v) formamide, 5×SSC, 1× Denhardt's solution, 20 mM dextran sulfate. Hybridized membranes were washed with 2×SSPE, 0.5% (w/v) SDS for 15 minutes at room temperature, 2×SSPE, 0.5% (w/v) SDS for 15 minutes at 65° C., and 0.2×SSPE, 0.2% (w/v) SDS for 15 minutes at 65° C. The washed filters were exposed to X-Ray film (XOMAT-AR, Kodak, Rochester, N.Y.) with an intensifying screen. Positive plaques were subjected to two additional rounds of screening to isolate single positive plaques. The cDNA inserts from positive plaques were excised from the X-vector as recombinant pBK-CMV phagemids. A full-length clone was completely sequenced by primer walking.

Expression of the V. planifolia OMT in Escherichia coli

The coding sequence of the OMT was amplified by PCR using oligonucleotides that introduced XhoI sites at the 5′ and 3′ ends. The PCR amplification product was separated on a 1% (w/v) agarose gel and the DNA band excised from the gel and extracted using a commercial kit (QIAquick Gel Extraction Kit, Qiagen). The PCR product was digested with XhoI and again gel purified. The digested PCR product was then ligated to the XhoI-digested dephosphorylated pET-15b expression vector (Novagen, Madison, Wis.) and transformed into ElectroMAX™ DH10B cells (Invitrogen) via electroporation. Plasmids from positive transformants were completely sequenced to confirm that no errors had been introduced through the PCR process. A plasmid containing the perfect OMT sequence was then transformed in BL21(DE3) cells (Novagen) for protein expression.

A BL21(DE3) OMT transformant was grown at 37° C. in LB with 50 μg ml⁻¹ ampicillin to OD₆₀₀=0.5. Protein expression was then induced by adding IPTG to 0.05 mM. Additional 50 μg ml⁻¹ ampicillin was also added and the cells grown overnight at 20° C. The cells were collected by centrifugation at 12,000 g for 15 min, lysed using BugBuster™ Protein Extraction Reagent (Novogen) and treated with Benzonase Nuclease (Novogen) according to the manufacturer's instructions. Cell debris was removed by centrifugation at 12,000 g for 20 min, the clarified lysate was applied to a His-Bind column (Novogen), and the expressed OMT protein eluted according to the manufacturer's instructions. The eluted protein was passed through a PD10 column (Amersham Pharmacia Biotech AB, Uppsala Sweden) equilibrated with the OMT assay buffer and concentrated 3-fold using Ultrafree-4 centrifugal filter units (Millipore Corporation, Bedford, Mass.). The concentrated protein was used for enzyme activity assays.

Antibody Production and Immunoblot Analysis

The purified recombinant protein was used for preparation of V. planifolia OMT-specific antiserum. The purified protein was mixed with an equal volume of Freund's complete (first injection) or incomplete (subsequent injections) adjuvant and was injected into the subscapular space of a rabbit. Three injections of about 100 μg of protein each were given at 4-week intervals.

For immunoblot analysis, proteins were extracted by homogenizing tissue samples in phosphate-buffered saline (1.5 mM NaH₂PO₄, 8.1 mM Na₂HPO₄, 145.5 mM NaCl) in a ratio of 0.4 g 800 μl⁻¹. The extracts were centrifuged to remove debris and the protein concentrations of the supernatants determined using the Bio-Rad protein assay reagent. Twenty μg of protein was mixed with an equal volume of 2× sodium dodecyl sulfate (SDS) sample buffer [2×: 125 mM Tris, pH 6.8, 4.6% (w/v) SDS, 10% (v/v) 2-mercaptoethanol, 20% (v/v) glycerol and 0.002% bromophenol blue (w/v). The proteins were transferred to nitrocellulose membranes (NitroPure, Osmonics, Westborough, Mass.) in 10 nM 3-(cyclohexylamino)-1-propane-sulfonic acid (CAPS), pH 11, 10% methanol (v/v). Processing and detection by chemiluminescence (Western Lightening Chemiluminescence Kit, Perkin Elmer Life Science) was according to the manufacturer's instructions.

### EXAMPLE 2

### Characterization of vpOMT Activity, Protein and cDNA

3,4-Dihydroxybenzaldehyde-O-methyltransferase Activity in V. planifolia Pods and Tissue Culture

A three-step pathway for vanillin biosynthesis from 4-coumaric acid has been proposed based on precursor accumulation and on feeding cell cultures of V. planifolia with the proposed precursors (Havkin-Frenkel et al., in: T. J. Fu, G. Singh, W. R. Curtis (Eds.), Plant Cell and Tissue Culture for the Production of Food Ingredients, Kluwer Academnic Press/Plenum Publishers, New York, 1999, pp 35-43). In this pathway 4-coumaric acid is first converted to 4-hydroxybenzaldehyde through a chain-shortening step. Hydroxylation at position 3 on the ring results in 3,4-dihydroxybenzaldehyde (also called protocatechuic aldehyde). The 3-hydroxyl group is then methylated producing vanillin. An enzyme from V. planifolia that catalyzes the chain-shortening step, 4-hydroxybenzaldehyde synthase, has been isolated as described hereinabove (see also, Podstolski et al., 2002, supra).

The proposed 3-step vanillin biosynthetic pathway postulates a 3,4-dihydroxybenzaldehyde-O-methyltransferase (DOMT) activity as the final step resulting in the production of vanillin. Green V. planifolia pods at different stages of development were obtained from Indonesia. Crude extracts of the inner region of the pods where vanillin is synthesized were assayed for DOMT activity by following the transfer of [¹⁴C] from radiolabelled SAM to 3,4-dihydroxybenzaldehyde. DOMT activity doubled between 3 and 5 months after pollination and was maintained at a similar level through 11 months after pollination (Table 2). The increase in DOMT activity at 5 months after pollination corresponded to the developmental stage when vanillin accumulation in the pods begins.

TABLE 2
DOMT and COMT activities (pkat mg⁻¹ protein)
in V. planifolia pods and tissue
culture extracts. Values presented are the means of duplicate assays.

| Sample | DOMT Activity | COMT Activity |
|---|---|---|
| | pkat mg⁻¹ | |
| Pods, crude extracts | | |
| Months after pollination | | |
| 3 | 0.37 | N.D.¹ |
| 5 | 0.90 | N.D. |
| 8 | 0.78 | N.D. |
| 11 | 0.80 | N.D. |
| Tissue culture | | |
| Crude extract | 0.96 | 0.79 |
| Adenosine column | 17.5 | 13.2 |

¹Not determined

Tissue cultures of V. planifolia have been established that accumulate vanillin and its proposed precursors, including 3,4-dihydroxybenzaldehyde (Havliin-Frenkel et al., 1996, Plant Cell Tiss Org Cult 45: 133-136). Crude extracts of the tissue cultures were found to have both DOMT and COMT activities (Table 2). With 3,4-dihydroxybenzaldehyde as the substrate, [¹⁴C]vanillin was identified as the product by co-migration with unlabeled standard vanillin on a TLC plate. Seventy-eight percent of the radioactivity present in the crude reaction product was recovered from the TLC plate at the position of authentic vanillin.

Partial Purification of DOMT Activity from V. planifolia Tissue Culture

Since the V. planifolia tissue cultures had DOMT activity at similar levels to the pods and were a convenient source of plant material, the first approach to characterizing the enzyme was to purify it from the tissue cultures. Affinity purification by binding to adenosine-conjugated agarose has been successful in purifying some OMTs. Both DOMT and COMT activities could be partially co-purified from the tissue culture crude extract by chromatography on an adenosine-agarose column (Table 2). SDS gel analysis of the active fractions revealed a major band at approximately 42 kD and a minor band at approximately 27 kD. COMTs from other species are in the range of 37.6-42.3 kD. The 42 LD band seen in the SDS gel of the active fractions appeared to be a single band and was likely the source of the O-methyltransferase activities. Peptide sequencing of the 42 kD band, however, revealed it was heterogeneous and no sequences similar to COMTs were obtained. Additional purification attempts were made using other column chromatography methods, but none were successful in separating the DOMT and COMT activities from each other.

V. planifolia O-methyltransferase cDNA Clone

To test whether the DOMT activity detected in V. planifolia tissue cultures originated from a multifunctional methyltransferase that could methylate both 3,4-dihydroxybenzaldehyde and caffeic acid, the inventors isolated a cDNA clone based on conserved sequences in COMTs from other species for expression in E. coli. Degenerate oligonucleotides based on the peptide sequences VLMESWY and HVGGDMF were used in PCR of a cDNA library prepared from the V. planifolia tissue culture. A 350 bp amplified band was cloned whose sequence was similar to COMTs from other plants. The PCR clone was used to screen the cDNA library and a full-length clone was obtained. A 365 amino acid protein with a molecular weight of 40,659 daltons was predicted from the cDNA sequence.

Similarity of the V. planifolia OMT to Other Sequences

The V. planifolia OMT amino acid sequence is similar to COMTs reported from other plant species but the level of identity is not high to any other sequences currently in the database. COMT sequences previously reported to be from V. planifolia (Xue and Brodelius; 1988, Plant Physiol. Biochem. 36:779-788) have been withdrawn from the NCBI database and now appear to actually be from Catharanthus roseus (Schroder et al., 2002, Phytochemistry 59: 1-8). Phylogenetic analysis comparing 19 similar methyltransferase sequences illustrates the relationship of the V. planifolia OMT sequence to methyltransferases reported from other species. The amino acid sequence of the V. planifolia OMT shows a similar level of divergence from the other monocot OMTs as from the dicot OMTs, perhaps reflecting its phylogenetic distance from the other reported monocot COMTs. V. planifolia is classified in the order Asparagales, whereas the other monocot species in'the COMT sequence comparison are in the order Poales.

Although there is considerable overall amino acid sequence variability among the monocot and dicot COMTs, all the residues identified from the crystal structure of the alfalfa enzyme as being involved in substrate binding or positioning (Zubieta et al., 2002, Plant Cell 14: 1265-1277) are generally well-conserved among most of the enzymes, including the V. planifolia OMT. The one nonconserved substrate-binding residue in the V. planifolia enzyme is N185 which is H183 at the corresponding position of the alfalfa enzyme. Amino acid residues at the relative position of the alfalfa substrate-binding residue 1316 exhibit considerable variation among the other COMT sequences.

Two tobacco COMTs that are quite different from each other have been reported and their substrate preferences have been compared (Maury et al., 1999, Plant Physiol. 121: 215-223; Pellegrini et al., 1993, Plant Physiol. 103: 509-517). The relative substrate preferences of tobacco class I COMT were similar to those of the alfalfa enzyme whereas tobacco class II COMT had no activity against caffeic acid and 5-OH-ferulic acid, but did have activity against 3,4-dihydroxybenzaldehyde (Maury et al., 1999, supra). Vanillin, the product of 3,4-dihydroxybenzaldehyde methylation, has been detected in tobacco and its accumulation was 10-fold higher in a phenylalanine ammonia-lyase overexpressing cell line. The tobacco class II COMT does differ from the alfalfa sequence at 5 of the conserved substrate binding residues, suggesting these differences may relate to the observed differences in substrate preferences.

Two barley sequences that are also quite different from each other have been reported as COMTs (Lee et al., 1997, DNA Seq. 7: 357-363; Sugimoto et al., 2003, Biosci. Biotechnol. Biochem. 67: 966-972). These two sequences stand out as different from the others in amino acid sequence comparisons in that there is sequence variation at a number of the conserved substrate-binding residues, and even in some catalytic sites, suggesting a closer evaluation of the activities and substrate preferences of those enzymes may be interesting. Barley EST sequences that are more closely related to the wheat sequence and which do have the conserved substrate binding residues have been reported (GenBank Accession Numbers AL5051022, AL504589, HVSMEn0023E17f, HVSMEn0007I18f, HVSMEn0023M14f, HVSSMEn0025G07f, and HVSMEn0009H02f).

Expression of V. planifolia OMT in E. coli

The protein encoded by the V. planifolia OMT cDNA was expressed as an N-terminal polyhistidine-tagged fusion in E. coli from the expression vector pET-15b and the recombinant protein purified by affinity chromatography. The expressed protein tended to rapidly accumulate in insoluble inclusion bodies, so conditions were developed using a low concentration of IPTG and low incubation temperature to allow accumulation of soluble OMT protein.

The kinetic parameters of the purified recombinant protein were determined with several phenolic and phenylpropanoid substrates (Table 3). The enzyme exhibited a preference for 5-OH-ferulic acid ethyl ester and caffeic acid ethyl ester, although these are unlikely to serve as substrates in vivo. Caffeoyl aldehyde and 5-OH-coniferaldehyde were preferred over 5-OH-ferulic acid, 3,4-dihyroxybenzaldehyde, or caffeic acid. In general, the relative substrate preferences for the V. planifolia enzyme were similar to those reported for alfalfa COMT (Parvathi et al., 2001, Plant J. 25: 193-202, which has been confirmed by down-regulation to be involved in S lignin biosynthesis (Guo et al., 2001; Plant Cell 13: 73-88). This suggests that the V. planifolia enzyme characterized here may also function primarily in the synthesis of lignin.

TABLE 3
Relative substrate preferences of V. planifolia recombinant OMT

| Substrates | Vmax/Km |
|---|---|
| 5-OH-Ferulic acid ethyl ester | 38.6 |
| Caffeic acid ethyl ester | 36.0 |
| Caffeoyl aldehyde | 28.4 |
| 5-OH-Coniferaldehyde | 19.7 |
| 5-OH-Ferulic acid | 9.6 |
| 3,4-Dihydioxybenzaldehyde | 2.0 |
| Caffeic acid | 1.9 |

V. planifolia OMT Expression in Different Tissues

Expression of the V. planifolia OMT in different tissues was evaluated by immunoblot analysis. As expected, the OMT protein detected in the tissue samples was slightly smaller than the purified recombinant His-tagged fusion protein. The highest OMT protein level was in the root and tissue culture samples, with a lower level in the stem sample. No immunoreactive band at the size of the OMT was detected in the leaf or pod samples. The origin of the higher molecular weight bands observed in the stem and leaf samples is not known. The lack of an immunoreactive band in the pod tissue was unexpected and surprising, since both the pods and tissue cultures synthesize vanillin and both had DOMT activity at similar levels (Table 2). These results suggest that the DOMT activities detected in these tissues originate from distinct enzymes that do not exhibit antibody cross reactivity. If this OMT is involved in the synthesis of vanillin it must be present in the pods at low levels that are not detectable by immunoblot analysis of proteins from crude extracts. Since DOMT activity was detectable in the pods, however, lack of an immunorective protein band suggests this OMT is not the main contributor to the observed activity. Although the V. planifolia OMT characterized here can convert 3,4-dihydroxybenzaldehyde to vanillin in vitro, the kinetic parameters and the tissue localization suggest its primary function is likely to be in lignin biosynthesis.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed:

1. A transgenic organism that produces vanillin when provided with caffeic acid or an esterified derivative thereof, the organism comprising expressible transgenes encoding:
a) a 3-O-methyltransferase that catalyzes methylation of caffeic acid to form ferulic acid; and
b) a chain-shortening enzyme that non-oxidatively converts ferulic acid to vanillin.

2. The organism of claim 1, which contains an endogenous esterase that hydrolyzes esters of caffeic acid.

3. The organism of claim 1, which further comprises an expressible transgene encoding an esterase that hydrolyzes esters of caffeic acid.

4. The organism of claim 1, which is a procaryote.

5. The organism of claim 4, which is Escherichia coli or Pseudomonas spp.

6. The organism of claim 1, which is a eucaryote.

7. The organism of claim 6, which is Pichia pastoris or Saccharomyces cerevisiae.

8. The organism of claim 1, wherein the 3-O-methyltransferase is from a plant source.

9. The organism of claim 8, wherein the plant source is selected from the group consisting of Catharanthus roseus, Clarkia breweri, Coffea canephora, Eucalyptus gunnii, Festuca arundinacea, Hordeum vulgare, Lolium perenne, Medicago sativa, Nicotiana tabacum, Ocimum basilicum, Populus tremuloides, Prunus amygdalus, Saccharum officinarum, Sorghum bicolor, Thalictrum tuberosum, Triticum aestivum, Vanilla planifolia and Zea mays.

10. The organism of claim 9, wherein the 3-O-methyltransferase is from Vanilla planifolia.

11. The organism of claim 1, wherein the chain-shortening enzyme is from a plant source.

12. The organism of claim 11, comprising a 4-hydroxybenzaldehyde synthase from Vanilla planifolia.

13. The organism of claim 1, wherein the chain shortening enzyme is from a bacterial source.

14. The organism of claim 13, comprising enoyl-SCoA hydratase/lyase.

15. A method for producing vanillin, which comprises:
a) providing a transgenic organism that produces vanillin when provided with caffeic acid or an esterified derivative thereof, the organism comprising expressible transgenes encoding:
i) a 3-O-methyltransferase that catalyzes methylation of caffeic acid to form ferulic acid; and
ii) a chain-shortening enzyme that non-oxidatively converts ferulic acid to vanillin;
b) culturing the transgenic organism in the presence of the caffeic acid or esterified derivative thereof, under conditions whereby the transgenic organism produces vanillin; and
c) recovering the vanillin from the culture.

16. The method of claim 15, wherein the organism contains an endogenous esterase that hydrolyzes esters of caffeic acid.

17. The method of claim 15, wherein the organism comprises an expressible transgene encoding an esterase that hydrolyzes esters of caffeic acid.

18. The method of claim 15, wherein the organism is a procaryote.

19. The method of claim 18, wherein the organism is Escherichia coli or Pseudomonas spp.

20. The method of claim 15, wherein the organism is a eucaryote.

21. The method of claim 20, wherein the organism is Pichia pastoris or Saccharomyces cerevisiae.

22. The method of claim 15, wherein the organism comprises a 3-O-methyltransferase from a plant source.

23. The method of claim 22, wherein the plant source is selected from the group consisting of Catharanthus roseus, Clarkia breweri, Coffea canephora, Eucalyptus gunnii, Festuca arundinacea, Hordeum vulgare, Lolium perenne, Medicago sativa, Nicotiana tabacum, Ocimum basilicum, Populus tremuloides, Prunus amygdalus, Saccharum officinarum, Sorghum bicolor, Thalictrum tuberosum, Triticum aestivum, Vanilla planifolia and Zea mays.

24. The method of claim 9, wherein the organism comprises 3-O-methyltransferase from Vanilla planifolia.

25. The method of claim 15, wherein the organism comprises a chain-shortening enzyme from a plant source.

26. The method of claim 25, wherein the organism comprises a 4-hydroxybenzaldehyde synthase from Vanilla planifolia.

28. The method of claim 15, wherein the organism comprises a chain shortening enzyme from a bacterial source.

29. The method of claim 28, wherein the organism comprises enoyl-SCoA hydratase/lyase.

30. The method of claim 15, comprising providing the organism with caffeic acid.

32. The method of claim 15, comprising providing the organism with a caffeic acid ester.

33. The method of claim 33, wherein the caffeic acid ester is one or more of cichoric acid, rosmarinic acid, chlorogenic acid, 1-caffeolylquinic acid or 1,5-dicaffeolylquinic acid.

34. An O-methyltransferase from Vanilla planifolia that catalyzes methylation of substrates selected from the group consisting of 5-OH-ferulic acid ethyl ester, caffeic acid ethyl ester, caffeoyl aldehyde, 5-OH-coniferaldehyde, 5-OH-ferulic acid, 3,4-dihydroxybenzaldehyde and caffeic acid.

35. The O-methyltransferase of claim 34, having an amino acid sequence at least 90% identical to SEQ ID NO:2.

36. The O-methyltransferase of claim 35, comprising amino acid SEQ ID NO:2.

37. An isolated nucleic acid molecule that encodes the O-methyltransferase of claim 34.

38. The isolated nucleic acid molecule of claim 37, which encodes a polypeptide having an amino acid sequence at least 90% identical to SEQ ID NO:2.

39. The isolated nucleic acid molecule of claim 38, which encodes a polypeptide having SEQ ID NO:2.

40. The isolated nucleic acid molecule of claim 39, having a sequence of SEQ ID NO:1.


SEQUENCE LISTING (2 sequences)

SEQ ID NO: 1 - DNA, 1219 bp, Vanilla planifolia
gaaatggctacatgggtggagcaccaacagcagcaaaatggatccaaggacgtggacgaggaggcgtgcatgtacgccatgcagttgtcgagcatggtcgtcctcccgatgacgcttagggtagccgtcgagctcggcatactcgaacaaatccaggccgggggcccagattcgtaccttactgccgaggatttggcggcgaggctcggcaactccaaccccttagctccggtcatgatcgagcggatcctgcgcctgctcaccagctactccatccttaacttcaccgacaccgtcgacggggagggtaggaccgtccggagctacggcgcggcgcatgtctgcaagtacctgactcccaaccaggacggcgtctccatggcgcctctcgtcctcatgaacacggataaggtccttatggagagctggtaccacatgaaggatgcagtgacaaatggtggaataccattcaatctagcatatgggatgacagcttttgagtatcatgggaaagatctaaggtttaataaggtgttcaacgagggcatgaagaacaactcgatcattataacgaagaagattttagagagatacaaaaggtttgaagatgtcaatgttttaattgatgttggtggtggaattggtggaactatcagtatgattactgcaaagtatccacatatacatgggattaattttgaccttcctcatgttgtttctgaagctccacctttccaaggggtagaacatgtcggtggaaacatgtttgaaagtgtccccattggtgatgcaatcttcataaagtggattcttcatgattggagtgatgagcattgtttgaagctcctaagaaattgtgcaaaatctttacctgacaaaggaaaagtcatagttgtggaatgcattcttcccgatgcacctttggtgacgccagaggctgaaggtgtctttcatttggacatgataatgttggctcacaatcctgggggaaaggagagaacaaagaaagagtttaaagaattggctatgctatctggtttctctaatttcaaggcactttttagttatgctaatgtttgggtcatggaattcaacaaatagtaatcttgaaactccatattgtgtggctaatttggatgctattttcacatgcaaggatgtggtgtctcttattatatgaatgtatgcattatttttcttaaaaaaaaaaaaaaaaaaa

SEQ ID NO: 2 - protein, 365 aa, Vanilla planifolia
MATWVEHQQQQNGSKDVDEEACMYAMQLSSMVVLPMTLRVAVELGILEQIQAGGPDSYLTAEDLAARLGNSNPLAPVMIERILRLLTSYSILNFTDTVDGEGRTVRSYGAAHVCKYLTPNQDGVSMAPLVLMNTDKVLMESWYHMKDAVTNGGIPFNLAYGMTAFEYHGKDLRFNKVFNEGMKNNSIIITKKILERYKRFEDVNVLIDVGGGIGGTISMITAKYPHIHGINFDLPHVVSEAPPFQGVEHVGGNMFESVPIGDAIFIKWILHDWSDEHCLKLLRNCAKSLPDKGKVIVVECILPDAPLVTPEAEGVFHLDMIMLAHNPGGKERTKKEFKELAMLSGFSNFKALFSYANVWVMEFNK